(19) United States
(12) Patent Application Publication
(10) Pub. No.: US 2006/0257846 A1
(43) Pub. Date: Nov. 16, 2006

(54) Method of assaying for high performance mammals

(75) Inventors: Stephen Christopher Bishop, Midlothian (GB); Elizabeth Janet Glass, Edinburgh (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Roslin Institute (Edinburgh)

(21) Appl. No.: 10/545,720

(22) PCT Filed: Feb. 16, 2004
(86) PCT No.: PCT/GB04/00563

§ 371 Date: May 24, 2006

(30) Foreign Application Priority Data
Feb. 15, 2003 (GB) 0303536.7

(51) Int. Cl. C12Q 1/00; G01N 33/567

(52) U.S. Cl. 435/4; 435/7.2

(57) ABSTRACT

A method of assaying for animals having a high innate immunity level by assessing the total white blood cell count of the mammal or at least one of the mammal's parents and/or the acute phase protein level of the mammal or at least one of its parents. Alternatively, genetic markers indicative of these values may be used. The values obtained are compared to equivalent measurements from other mammals of the same breed. Values higher than mean equivalent measurements from mammals of the same breed indicate a high innate immunity level which is associated with a high performance.

The present invention relates to a method of assaying for high performance mammals through assessment of the innate immunity of the mammal, or one of the mammal's parents. Particularly but not exclusively the assay involves a method of assaying for high performance pigs.

The pig breeding industry has traditionally concentrated on production traits such as growth rate, carcass characteristics and litter size in its breeding programmes. Breeding programmes have placed less emphasis on the potential benefits that may be obtained from selecting pigs that show a greater degree of disease resistance. Benefits to the pig industry alone include: reducing the cost of controlling disease and treating sick animals, lessening the impact of acute infections in a pig herd and, in the case of chronic infections, healthier and more productive pigs. In addition to missing out on these benefits, current selection programmes which concentrate on production traits result in unpredictable correlated responses in disease resistance, and this poses a risk which must be addressed.

In a situation where, there is a ubiquitous disease of particular importance and it is known that resistance to this disease has an inherited component, animals may be selected for resistance to the specific disease. Given that protection against different diseases involves different immune mechanisms, e.g. antibody, cell mediated and innate immune responses, it should be recognised that this strategy may not improve resistance to diseases other than the specific disease selected against.

In contrast the present invention provides a method of selecting animals for “generalised immunity”, i.e. a generally enhanced immune responsiveness to a variety of disease challenges. The principle is that animals having enhanced generalised immunity have a greater degree of resistance against a variety of diseases and thus the diseases to be protected against do not have to be identified. This is a particularly important consideration as sub-clinical infections play an important role in poor performance.

The aim of improving “generalised immunity” is to produce animals more able to respond to a variety of disease challenges and is therefore an appropriate strategy for breeding programmes with a main focus on productivity. Breeds differ in their general disease resistance and hardiness, with the Duroc being an example of a breed with superior hardiness (as evidenced by their inclusion in outdoor production systems).

Edfors-Lilja et al. (“Mapping Quantative Trait Loci for Immune Capacity in the Pig.” The American Association of Immunologists 1998 22:1767) investigated the differences in total leukocyte counts, mitogen-induced proliferation, prevaccination Ab levels to E. coli and Ab response to E. coli 0149 Ag in domestic and wild pigs. It was postulated that these values reflected immune capacity traits in pigs.

Edfors-Lilja et al. (“Mapping quantitative trait loci for stress induced alterations in porcine leukocyte numbers and functions”. Animal Genetics, 2000, 31, 186-193) identified four quantitative trait loci reflecting porcine immune functions and compared these values in wild and domestic pigs This document made no teaching or suggestion that quantitative trait loci may be compared between pigs of the same breed to identify individual high performance pigs.

Henryon et al. (“Genetic variation for Total and Differential Numbers of Leukocytes exists in Growing Pigs”. 7^{th} World Congress on Genetics Applied to Livestock production, Aug. 19-23, 2002, Montpellier, France. Communication 13-02) postulate that relative white blood cell counts (i.e. leukocytes) may indicate resistance to clinical and sub-clinical disease. Henryon et al. do not however present a within-sample repeatability. Furthermore they do not provide any data to back their postulation. The methodology proposed is therefore not backed by data and no indication is given of its reliability for use in the field.

WO 94/14064 refers to the use of an index of antibody, cell mediated and immune responsiveness, in the within-breed genetic selection of pigs. There was no consistent evidence for improved disease resistance in the line selected for improved immune responsiveness.

In addition, WO 94/14064 teaches methods that only include measures of immune response, that is the immune system of the animal is artificially challenged and its response determined.

The present invention investigates and quantifies generalised immunity in genetically diverse populations of mammals, particularly pigs. The present invention identifies and focuses on components of innate immunity that are without any treatment or challenge. The benefits of this approach are (i) it focuses on the primary determinant of immune response (innate immunity) and (ii) it uses measurements which do not require the animals to be challenged and thus can easily be incorporated into breeding programmes. The present invention concentrates on innate immunity as, although different diseases require different adaptive immune responses for protection, all pathogens switch on innate defences which are always poised ready for rapid action. Also, innate pathways play an important role in regulating specific immunity. Thus, by increasing the innate immunity of a group of mammals the general disease resistance of the group of mammals is improved. Increased resistance to a variety of pathogens will result in animals that suffer less from subclinical disease and consequently have improved performance characteristics.

The present invention provides a method of assaying for the innate immunity level of a mammal, said assay comprising the steps of;
(i) assessing the total white blood cell count of the mammal or at least one of its parents and/or the acute phase protein level of the mammal or at least one of its parents and/or the incidence of genetic markers indicative of one or more of these measurements;
(ii) comparing the measurements so obtained with equivalent measurements from other mammals of the same breed wherein measurements higher than mean equivalent measurements from mammals of the same breed indicate a high innate immunity level.

Preferably a high innate immunity level is associated with increased feed to weight efficiency, increased resistance to pathogenic infection and/or decreased deleterious or pathogenic consequences of infection.

Characteristics of increased innate immunity, such as increased resistance to pathogens and high feed to weight efficiency are associated with high performance mammals. Efficiency may be measured by calculating the weight gain of the mammal divided by the food consumed. Preferably high performance mammals have the characteristic of increased lean gain under restricted or ad libitum feeding. Clearly this method could be used to select for either high performance mammals suitable for breeding, or equally to identify low performance animals which may be excluded from the breeding herd.

Preferably the mammal is a pig.

Where the parent of the animal of interest is tested (in preference to the animal itself), for convenience the parent may by the sire. However testing of the dam is not excluded. Optionally both parents may be tested.

In one embodiment the total white blood cell count is the parameter tested.

In a different embodiment the acute phase protein level is the parameter tested.

In a further embodiment the incidence of genetic markers indicative of the total white blood cell count of the mammal is tested. In a further embodiment the incidence of genetic markers indicative of the acute phase protein levels of the mammal is tested. Alternatively in a different embodiment the incidence of genetic markers indicative of the total white blood cell count and acute phase protein level of the mammal is tested.

In one embodiment the method of assaying the innate immunity and hence the performance of mammals comprises the steps of testing the white blood cell count of the mammal or at least one of its parents and testing the acute phase protein levels of the mammal or at least one of its parents, and comparing the results to the mean of the equivalent measurements for that breed, wherein a white blood cell count and an acute phase protein level higher than the mean level for mammals of the same breed is indicative of a high innate immunity level. Suitably the acute phase protein is alpha-1 acid glycoprotein (α₁-AGP), serum amyloid A (SAA) or haptoglobin. Preferably the acute phase protein is α₁-AGP and/or SAA.

The acute phase protein may be measured in blood samples taken from the mammal or at least one of its parents and may conveniently be taken at the same time as these for white blood cell counts and measured using, for example, radial immunodiffusion assays.

Genetic markers associated with a high white blood cell count and/or a high acute phase protein level can be used instead of (as a surrogate for) the actual immune measurements. Preferably the method of assaying for the innate immunity comprises the step of assessing the incidence of genetic markers indicative of the white blood cell count of at least one of the mammal's parents and the incidence of genetic markers indicative of the acute phase protein level of at least one of the mammal's parents. Alternatively the method may comprise assaying the incidence of genetic markers indicative of the white blood cell count of the mammal and the incidence of genetic markers indicative of the acute phase protein levels of the mammal.

Advantageously more than one white blood cell count and/or assessment of the acute phase protein level is taken at spaced intervals.

Advantageously the method of assaying for high performance mammals comprises the step of assessing the proportion of mononuclear cells positive for NK (Natural Killer), B cell and monocyte markers. These measurements may be considered to be predictive of the current infection status of the mammal. As the proportion of NK cells, B cells and monocytes increases, the innate immunity and performance levels of the individual mammal tends to decrease.

The proportion of mononuclear cells positive for NK, B cell and monocyte markers can be assayed by identifying, categorising and enumerating blood mononuclear cell subpopulations and measuring the number categorised as being NK cells and/or B cells and/or monocytes, and expressing each of these categories as a proportion of the overall mononuclear cell population.

Advantageously the measurements taken to assess the innate immunity of the mammal are compared within a single sex, on animals exposed to the same environment, for example by being housed on the same farm. Suitably all measurements compared are extracted from the mammals within 24 hours of each other, preferably within 1 hour of each other. The samples are suitably assayed on the same day or with minimal delay from extraction of the sample from the mammal. Advantageously more than one sample is tested from each animal at spaced intervals.

The blood sample is typically mixed with an anti-coagulant such as EDTA, and used to evaluate the total white blood cell counts and/or the levels of acute phase proteins. Where the blood sample is used to evaluate the levels of acute phase proteins, the blood sample may be centrifuged, suitably at 1000 g, suitably for approximately 10 to 20 minutes to separate plasma. Plasma separation is preferably carried out within eight hours of blood collection.

Suitably the method also comprises the step of taking samples of blood from mammals being of the same breed, being housed under the same conditions where all samples compared are taken at approximately the same time, preferably within 24 hours of each other, suitably 5 hours or less, advantageously within 1 hour of each other. Suitably six mammals or more are tested to calculate the mean values, typically ten mammals or more, preferably twenty mammals or more, more preferably fifty mammals or more.

Preferably the method of assaying the innate immunity levels of mammals comprises the steps of;
i) assessing the total white blood cell counts of the mammal or at least one of its parents and/or the acute phase protein levels of the mammal or at least one of its parents and/or the incidence of genetic markers indicative of one or more of these measurements;
ii) comparing the measurements obtained with the mean levels of equivalent measurements for animals of the same breed as the animal tested.

The present invention also provides a method of assaying for a breed of mammal which exhibits high innate immunity levels, said method comprising the steps of assaying for the performance of mammals within the breed according to the method described above, calculating an average innate immunity level of mammals within the breed and comparing the average innate immunity levels to equivalent values obtained for other breeds of the mammal.

According to a further aspect of the present invention there is provided an assay to create a generalised immunity index for a mammal by testing the total white blood cell counts of the mammal and assessing the proportion of mononuclear cells positive for NK, B cell and monocyte markers and combining these values.

The generalised immunity index may be calculated using the following formula;
Index=WBC/(s.d. WBC)+(NK prop)/(s.d. NK prop)+(B prop)/(s.d. B cell prop)+(Monocyte prop)/(s.d. monocyte prop).

Where—“WBC” is the total white blood cell count, “s.d.” is the standard deviation of a variable and “prop” means the proportion of mononuclear cells positive for a certain marker.

Higher generalised immunity index values are associated with genetically higher performance mammals.

The generalised immunity index is reflective of the health, and individual productivity of the mammal (in terms, for example of its feed:lean weight conversion).

The present invention also provides a kit for assessing the innate immunity levels of a mammal, said kit comprising means for testing the total white blood cell counts and/or acute phase protein levels and/or the incidence of genetic markers indicative of one or more of these measurements.

In one embodiment of the present invention the kit comprises means for testing the total white blood cell count.

In a different embodiment of the present invention the kit comprises means for testing the acute phase protein levels.

Preferably the kit also includes means for comparing the values obtained with a standard being the mean values for equivalent measurements for mammals of the same breed as the mammal being tested thereby determining the innate immunity level for said mammal.

The present invention also provides a kit for assessing the gereralised immunity index of an animal, said kit comprising means for testing the total white blood cell count of the mammal and the proportion of mononuclear cells positive for NK, B cell and monocyte markers, means for combining the total white blood cell count and the proportion of mononuclear cells positive for NK, B cell and monocyte markers and means for comparing these values with a standard being the mean values for mammals of the same breed as the mammal being assayed thereby determining the generalised immunity index value for said mammal.

Specific Measurements Investigated

From the large numbers of potential measurements, assays for the following categories of measurements were found to be of particular utility in assaying for genetically high performance mammals;

I Total Blood Cell Counts

The total white blood cell count of the mammal or its sire may be evaluated, where a high total white blood cell count is associated with high performance. In particular a high correlation has been noted between a high total white blood cell count of the sire and high performance progeny. The measurement of the total white blood cell count may be performed by counting the number of white blood cells using a haemocytometer, and expressing numbers as 10⁶ per ml.

II Alpha-1 Acid Glycoprotein

Plasma alpha-1 acid glycoprotein may be measured by a commercially available radial immunodiffusion assay, in which alpha-1 acid glycoprotein reacts with antiserum specific to alpha-1 acid glycoprotein leading to the formation of a visible precipitation ring. Alpha-1 acid glycoprotein concentration is directly proportional to the area of the precipitation ring. Furthermore, the following measurements were found to be of particular utility in developing an index of generalised immunity;

III Proportions of Mononuclear Cells Positive for NK, B Cell and Monocyte Markers

The proportions of mononuclear cells positive for NK, B cell and monocyte markers may be evaluated using appropriate monoclonal antibodies such as MIL-4 (isotype IgG1) (CD11R1, NK cell specific), K139 E1 (isotype IgG2a) which binds to the anti-porcine immunoglobulin light chain on B cells and 74-22-15 (isotype IgG2b) which binds to the SWC3a antigen on monocytes. Mononuclear cells may be incubated with the monoclonal antibodies for 30 minutes on ice and washed. Phycoerythin- or FITC-conjugated goat anti-mouse IgG1, IgG2a or IgG2b may be added to detect bound monoclonal antibodies of matching isotype. Typically, 10,000 fluorescent labelled cells are analysed by flow cytometry, with linear amplification of the forward and side scatter and with logarithmic amplification of the fluorescent signal.

An effective method of assaying for high performance mammals is disclosed as well as an index of generalised immunity, having an emphasis on traits of the innate immune response.

The attributes of these measurements are:
(i) they can be measured on a single blood sample taken from an unchallenged animal;
(ii) it is technically possible to do them on relatively large numbers of animals;
(iii) they are accurately measured and repeatable across time;
(iv) measurements on groups of animals are consistent across different sampling days;
(v) they are heritable;
(vi) they predict performance of mammals caused by both the genetics and/or environment of the mammal.

In terms of general summary of the properties of the generalised immunity index:
(i) white blood cell numbers are important, primarily, as they are genetically related to the efficiency of growth, e.g. lean gain under restricted feeding, and thus the performance of the mammal;
(ii) the proportions of mononuclear cells positive for NK, B cell and monocyte markers are important, primarily, as they are predictive of performance at the level of the individual mammal. They appear to be diagnostic of individual animal health levels, being environmentally related to performance.

This information may be used in two ways (as described above):
(i) The method of assaying for high performance mammals may be used to correct performance for the effect of any environmental challenges; or
(ii) the index of generalised immunity may be decomposed (e.g. by BLUP) into a genetic and environmental component. The environmental component can then be used to pre-correct performance traits for environmental challenge effects, and the genetic component used along with the corrected performance traits in a selection index describing overall performance.

The method of assaying for high performance mammals hereinbefore described enables pig breeders to (i) improve performance and (ii) deal with the genetic/environmental (GxE) problem in which pigs selected under high health status conditions disappoint when they are evaluated under ‘dirtier’ commercial conditions.

The possible use of genetic markers is particularly attractive under commercial conditions.

Potentially, markers may increase the accuracy of selection and make results independent of measurement environment.

The present invention will now be described by way of example only.

### EXAMPLE 1

Experimental Protocols

Demonstration of Genetic Influences on Immune Measurements

Pig Populations

Pigs studied were from the Edinburgh “Lean Growth” selection population and were of the “Large White” breed. In particular, the pigs in this study were derived from lines of pigs previously selected for either high or low lean growth under restricted feeding (the abbreviation LGR—Lean Growth Restricted feeding—will subsequently be used to describe these pigs). Lines with low vs high performance characteristics rate were compared. These pig populations differ in their growth rate and carcass lean content. When available, unselected control line pigs were also studied.

Measurement Strategy

The pigs were subjected to a standard performance test from 14 to 24 weeks of age, with individual growth rates and food intake collected. Blood samples were then collected at mid-test (18 weeks of age) and at the end of test (24 weeks of age), and assays performed.

The key to immunological measurements being of use within a generalised immunity framework is their repeatability. There are two components to repeatability:

I) the accuracy of the measurement and

II) the stability of the measurement across time.

The accuracy of the measurement may be assessed from the similarity between replicate measurements taken on the same blood sample, i.e. the within-sample repeatability. Values approaching 1.0 are desirable. The stability of measurements across time, i.e. the across-time repeatability, describes the degree to which measurements are generally descriptive or are specific to an animal on a given day. The across time repeatability is also an upper limit to the heritability. Arbitrarily we would wish across time repeatabilities to be in excess of 0.4-0.5.

Within-sample and across-time repeatabilities for total and differential white blood cell counts were estimated from duplicated assays performed on two blood samples per pig, taken one week apart, i.e. 4 measurements per pig. The results are indicative of the repeatability, and hence suitability of these measurements. Results of the repeatability studies are shown in Table 1. Also shown in Table 1 are the repeatabilities for acute phase proteins (alpha-1 acid glycoprotein), estimated from duplicated samples taken 6 weeks apart.

TABLE 1
Repeatability Analyses for each assay.

| Total & Differential | Within-sample | Across-time |
|---|---|---|
| White Blood Cell Counts | Repeatability | Repeatability |
| No. White Blood Cells | 0.98 | 0.50 |
| Neutrophil Count | 0.96 | 0.17 |
| As % of total WBC | 0.94 | 0.08 |
| Basophil Count | 0.23 | 0.10 |
| As % of total WBC | 0.48 | 0 |
| Eosinophil Count | 0.88 | 0.76 |
| As % of total WBC | 0.96 | 0.96 |
| Monocyte Count | 0.43 | 0.43 |
| As % of total WBC | 0.59 | 0.24 |
| Lymphocyte Count | 0.95 | 0.55 |
| As % of total WBC | 0.86 | 0 |
| Alpha-1 acid glycoprotein | 0.99 | 0.64 |

The measurement strategy performed on the pigs is summarised in Table 2. Suffixes 1 and 2 are used to specify groups of pigs, group 2 pigs are the next generation from the group 1 animals. For “line”, H=high, C=control, L=low, i.e. high refers to the high performance pig line. Pigs of both sexes were measured.

TABLE 2
Experimental design and measurement strategy.

| Population | LGR1 | LGR2 |
|---|---|---|
| Lines Tested | H, C, L | H, L |
| Stage of Test | End | Mid, End |
| No. of Pigs | 48 | 30 |
| No. of Measures | 48 | 60 |

White Blood Cell Protocols

WBC analysis was performed by counting the number of leukocytes using a haemocytometer, and expressing numbers as 10⁶ per ml. For leukocyte differentiation, blood smears were stained with Leishman stain and classified as lymphocytes, neutrophils, monocytes, eosinophils and basophils on the basis of morphology; numbers were again expressed as 10⁶ per ml.

Acute Phase Proteins Protocols

The acute phase protein measurements (alpha-1 acid glycoprotein) were measured on pig blood samples taken at the same time as those for white blood cell counts. Plasma alpha-1 glycoprotein was measured by a commercial radial immunodiffusion assay in which alpha-1 acid glycoprotein reacted with specific antiserum to alpha-1 acid glycoprotein leading to the formation of a visible precipitin ring and alpha-1 acid glycoprotein concentration was measured as being directly proportional to the area of the precipitin ring.

Mononuclear Cell Protocols

Mononuclear cells were isolated from the same blood samples as the white blood cells. The proportions of mononuclear cells positive for NK, B cell and monocyte markers were evaluated using the following monoclonal antibodies: MIL-4 (isotype IgG1) (CD11R1, NK cell specific), K139 E1 (isotype IgG2a) which binds to the anti-porcine immunoglobulin light chain on B cells and 74-22-15 (isotype IgG2b) which binds to the SWC3a antigen on monocytes. Mononuclear cells were incubated with the monoclonal antibodies for 30 minutes on ice and washed. Phycoerythin- or FITC-conjugated goat anti-mouse IgG1, IgG2a or IgG2b were added to detect bound monoclonal antibodies of matching isotype. Typically, 10,000 fluorescent labelled cells were analysed by flow cytometry, with linear amplification of the forward and side scatter and with logarithmic amplification of the fluorescent signal.

Results

Summary Statistics for Immunological Measurements of Entire Population

Summary statistics for some of the immunological measurements are presented below.

In addition to fitting fixed effects of sex and population/line, a random effect for day of sampling (nested within population) was also fitted, using a statistical technique known as residual maximum likelihood (REML). This between-day variation indicates the consistency of the measurement, ie. the degree to which measurements for a group of pigs jump about over time due to unspecified factors—in other words the reliability of measurements on a group of animals at a particular time. To summarise this information a parameter termed “Constancy” was calculated as [1-σ²(sampling day)/σ² (sampling day)+σ² (residual))], where σ² signifies a variance component. If the variation between days is similar to that which might be predicted from the normal variation between animals (ie. σ² (residual)), then the sampling day variance tends to zero and the constancy parameter tends to 1.0. If the measurements for groups of animals fluctuate considerably, then the constancy parameter becomes very small. For comparison, the constancy parameters for the performance test traits were generally greater than 0.8.

White Blood Cell Counts

Summary statistics for white blood cell counts are shown in Table 3. The standard deviation (s.d.) value is σ (residual). The correlations between measurements at mid and end of test for individual animals are perhaps lower than expected. Repeatability analyses found correlations between measurements taken one week apart to be 0.50 for total WBC; thus, the further apart in time measurements are taken, the lower the correlation.

TABLE 3
Summary statistics for total and differential WBC
(10⁶ cells/ml), at mid and end of test.

| | Total | | | | | |
|---|---|---|---|---|---|---|
| | WBC | Neutrophils | BasoPhils | Eosinophils | Monocytes | Lymphocytes |
| End Test | | | | | | |
| Mean | 32.6 | 8.48 | 0.20 | 0.86 | 1.74 | 21.33 |
| s.d. | 8.3 | 3.87 | 0.14 | 0.45 | 0.63 | 5.39 |
| Constancy | 0.98 | 0.86 | 0.98 | 1.00 | 0.75 | 1.00 |
| Mid Test | | | | | | |
| Mean | 32.8 | 10.30 | 0.22 | 0.68 | 1.92 | 19.50 |
| s.d. | 7.2 | 4.05 | 0.16 | 0.49 | 0.58 | 5.40 |
| Constancy | 0.72 | 0.81 | 0.97 | 0.82 | 0.68 | 0.88 |
| Correlation | 0.26 | 0.24 | 0.03 | 0.27 | 0.18 | 0.14 |
| (mid, end) | | | | | | |

Equivalent alpha-1 acid glycoprotein results were, Mid test: mean=436 μg/ml, s.d.=167 μg/ml, constancy=0.91; End Test: mean=261 μg/ml, s.d.=89 μg/ml, constancy=0.93.

Performance Traits

Summary statistics for performance traits are shown in Table 4. Efficiency is expressed as gain/food—this trait was normally distributed and easily interpretable insofar as larger values indicate better values. The constancy values and the correlations between performance in the two halves of the test period were generally similar to those for the immune measurements. This gives confidence that the immunological measurements are at least as reliable as the performance test traits.

TABLE 4
Summary statistics for performance traits

| | Daily gain | Daily FI | Gain/Food |
|---|---|---|---|
| | (kg) | (kg) | (kg/kg) |
| Whole Test | | | |
| Mean | 0.819 | 2.26 | 0.365 |
| s.d. | 0.092 | 0.25 | 0.027 |
| Constancy | 0.84 | 0.79 | 0.97 |
| Part-test means | | | |
| Start-mid | 0.782 | 1.95 | 0.402 |
| Mid-end | 0.859 | 2.57 | 0.336 |
| Correlation (mid, end) | 0.30 | 0.63 | 0.23 |

Statistics for Immunological Measurements in Particular Lines

Line means were estimated by analysing all data for each particular trait simultaneously, fitting sex and population/line as fixed effects and day of measurement within population as a random effect, using REML. Standard errors of line means and standard errors of differences, for significance testing, were constructed from the variance/covariance matrix of the line means.

Line Means for Total White Blood Cell Counts

Line means for total white blood cell counts are shown in Table 5. Values in parentheses following each mean are standard errors of the estimated means. Sed is the standard error of the difference against which the H-L difference is tested (**=1% significance levels, *=5% significance level). To help interpretation, significant results are shown in bold. - indicates that the test was not carried out for these animals.

Large and consistent differences in white blood cell numbers are seen between the H and L lines, at both stages of the test, with limited data suggesting the difference is symmetric about the control line. Consistent selection line differences indicate that white blood cell numbers are heritable and genetically correlated with the selection criterion.

TABLE 5
Line means for total white blood cell counts (10⁶ cells/ml),
(** = P < 0.01, * = P < 0.05)

| | LGR1 | LGR2 |
|---|---|---|
| End Test | | |
| H | 40.2(2.0) | 39.8(2.4) |
| C | 34.6(2.7) | — |
| L | 28.2(1.9) | 27.2(2.1) |
| H − L | 12.0** | 12.6** |
| Sed | 2.40 | 2.72 |
| Mid Test | | |
| H | — | 31.8(3.2) |
| C | — | — |
| L | — | 24.3(2.9) |
| H − L | — | 7.5* |
| Sed | — | 2.92 |

The H and L LGR lines have essentially been selected for changes in efficiency. Thus, the high (H) line has been selected to minimise wasteful metabolic effort. The presence of elevated white blood cells in the blood may be an indicator of the capability to respond efficiently to background infections. The impact of background infections is minimised by appropriate production of white blood cells—the cost of producing these cells is more than outweighed by the benefits that they confer. Likewise, part of the low (L) line response in becoming less efficient may be due to not having the ability to respond appropriately to background challenges. WBC counts are indicative of animals' previous challenges by infectious organisms and also indicative of their ability to cope with such challenges. All pigs in this study were housed together and hence faced the same challenge. Therefore, these WBC counts are indicative of their ability to cope and perform in a moderately infectious, ie “commercial” environment. These results indicate that having higher WBC counts is a mechanism by which selected pigs have been able to be more efficient within a “commercial” environment. These results indicate that selection using WBC counts or WBC QTL is a technique that can be used within a specific-pathogen-free environment to genetically improve performance and efficiency of progeny performing in a commercial environment.

Line Means for Acute Phase Proteins

Line means and differences for acute phase proteins between the High (H) and Low (L) lines for “lean growth under restricted feeding” lines (LGR1 and LGR2) are shown in Table 6. Sed is the standard error of the difference against which the H-L difference is tested (**=1% significance levels, *=5% significance level). For ease of reference, significant results are shown in bold. - indicates that the test was not carried out for these animals.

TABLE 6
Line means for alpha-1 acid glycoprotein (μg/ml),
(** = P < 0.01, * = P < 0.05)

| | LGR1 | LGR2 |
|---|---|---|
| Mid Test | | |
| H | — | 630.9 |
| L | — | 363.3 |
| H − L | — | 267.6** |
| Sed | — | 64.8 |
| End Test | | |
| H | 318.8 | 314.7 |
| L | 229.5 | 214.7 |
| H − L | 89.3* | 100.0** |
| Sed | 32.8 | 34.6 |

The interpretation of these results is the same as for the white blood cell counts. The H and L LGR lines have essentially been selected for changes in efficiency. Thus, the high (H) line has been selected to minimise wasteful metabolic effort. The presence of elevated acute phase protein levels may be an indicator of the capability to respond efficiently to background infections. The impact of background infections is minimised by appropriate production of acute phase proteins—the cost of producing these is more than outweighed by the benefits that they confer.

These results demonstrate that acute phase protein levels are heritable and genetically correlated with the lean gain under restricted feeding. Therefore, selection using acute phase protein levels is a technique that can be used within a specific-pathogen-free environment to genetically improve performance and efficiency of progeny performing in a commercial environment.

In summary, white blood cell counts and acute phase protein levels are consistent and significant predictors of performance genotype. Our results thus verify that innate immunity is critical and, furthermore, can be improved by selection within current breeds.

Immunological Traits as Predictors of Performance Traits for Individual Animals.

The line means presented above describe genetic relationships between specific selection strategies and immunological measurements. Significant results indicate that immunological measurements are heritable and related to that particular selection criterion. However, acting at the group mean level on pigs in the same environment, they only indicate genetic relationships. They give no information on the relation between the immune measurement and performance for the individual pig, i.e. they do not help to explain the performance or health status of individual pigs. This can be achieved by regressions of performance traits on immune traits, after removing genetic effects of selection line or breed, i.e. by looking at the within-line relationship between performance and immune measures. This regression will largely (but not entirely) describe environmental relationships between traits.

Regressions of performance traits on white blood cell numbers were generally small and not significant. Other factors in the model were sex, population/line and day of measurement.

It was found that the proportions of mononuclear cells that were positive for NK, B cell or monocytes markers (referred to as NK cells, B cells or monocytes) were predictive of performance. Regressions of performance traits on each of these measures are shown in Table 7.

TABLE 7
Regressions (×10³) of performance test traits on
the proportions of mononuclear cells positive for
NK, B cell or monocytes markers, measured at Mid
Test and End Test.

| | Daily Gain | Daily FI | Gain/Food |
|---|---|---|---|
| End-Test | | | |
| NK cells | −926.307 ± ** | −452 ± 886 | −341 ± 81** |
| B cells^{a} | −3450 ± 1960 | −15880 ± 5670** | 791 ± 531 |
| Monocytes | −640 ± 276* | −1063.42 ± 798 | −137 ± 75 |
| Mid-Test | | | |
| NK cells | −616 ± 244** | −1060 ± 711 | −139 ± 70** |
| B cells^{a} | −1770 ± 1890 | −5540 ± 5540 | 2810 ± 5370 |
| Monocytes^{a} | 3140 ± 1700 | 4800 ± 5180 | 5140 ± 4260 |

Performance traits describe the whole performance test.
(** = P < 0.01, * = P < 0.05).
Superscript^{a} indicates measurement square root transformed prior to analyses.

As proportions of NK cells, B cells and monocytes increase, performance of the pig tends to decrease, with all statistically significant regressions being negative, suggesting that these measurements are predictive of the current infection status of the animal.

The Index of Generalized Immunity

An index of generalized immunity was constructed, by combining the traits most significantly related to performance—in this case white blood cell count as an indicator of performance genotype and the proportion of NK cells, B cells and monocytes, as indicators of current infection status. Each trait was weighted by the standard deviation. Thus, for measurements taken at the end of the test period the index, which may be derived from single blood sample, was:
Index_{end}=WBC/8.3+(NK cell prop./3.03)+(B cell prop./3.71)+(monocyte cell prop./3.30)

A comparable index for measurements taken mid test was:
Index_{mid}=WBC/7.2+(NK cell prop./3.82)+(B cell prop./5.68)+(monocyte cell prop./3.98).

The denominators in these formulae are the standard deviations of each respective trait. Different data sets will clearly result in different standard deviations and therefore different formulae. Line means for the end and mid test indexes for the LGR2 population are shown in Table 8. These values are dimensionless. The constancy of the end of test index was 0.90, although the mid test index value was only 0.58. For the end test index, highly significant line differences are seen. Higher index values were associated with the biologically higher performing lines. This index is thus heritable and genetically correlated with biologically important variables. Significant differences were also seen in the mid test index. The correlation between the mid and end test indexes was 0.45.

TABLE 8
Line means for the Generalised Immunity index, at
end and mid test, (** = P < 0.01, * = P < 0.05).

| | LGR2 |
|---|---|
| End Test | |
| H | 18.0(0.81) |
| L | 14.4(0.70) |
| H − L | 3.63** |
| Sed | 0.92 |
| Mid Test | |
| H | 15.4(0.92) |
| L | 13.5(0.85) |
| H − L | 1.90* |
| Sed | 0.71 |

Regression coefficients of performance traits describing the whole test on the two indexes are shown in Table 9, along with corrected R² values for the statistical model with and without index. Other factors in the model were sex, population/line and day of measurement. With the exception of the regression of gain/food on the mid test index, where significance just failed to reach the 5% level, all regression coefficients were highly significant. Moreover, all regressions were in the biologically correct direction, i.e. negative. Furthermore, adding the index to the regression equations explaining each performance trait substantially reduced the residual standard deviation, this improving the fit of the model and hence the R² value—in all cases except for the regression of gain/food on the mid test index. Therefore, at the individual pig level, both indexes appear to be serving as a diagnostic of the individual health, and hence individual productivity.

TABLE 9
Regressions (×10³) of performance traits for the
whole performance test on the mid and end of test
generalised immunity index, and corrected R² values
with and without the index, (** = P < 0.01, * = P < 0.05).

| | Daily Gain | Daily FI | Gain/Food |
|---|---|---|---|
| Mid-Test | | | |
| Regression | −20.2 ± 4.8** | −40.9 ± 13.8** | −2.63 ± 1.36 |
| R² without | 0.35 | 0.34 | 0.42 |
| R² with | 0.49 | 0.42 | 0.44 |
| End Test | | | |
| Regression | −19.6 ± 3.5** | −32.4 ± 10.9** | −3.26 ± 1.07** |
| R² without | 0.35 | 0.38 | 0.49 |
| R² with | 0.51 | 0.43 | 0.54 |

To summarise the properties of the generalised immunity index:
it is consistent across day of measurement, as consistent as performance traits;
it is heritable;
it is genetically correlated with (desirable) performance attributes, i.e. lean growth under restricted feeding;
at the individual animal level it appears to be diagnostic of the health status of that pig, insofar it is predictive of performance: as the index goes down performance goes up.

These conclusions hold for both the end and mid test indexes. However, the results, including the constancy values, would suggest that the end of test index is the more reliable and effective index.

The index as it stands, i.e. as a summary of several traits, raises an apparent conceptual difficulty that must be explained, along with the solution to this problem. The apparent problem is that the genetic and environmental properties of the index conflict with each other. Genetically, improved index values point towards enhanced performance—pigs with higher index values and immune measures of this type will be better equipped genetically to withstand environmental (pathogen) challenges, and hence perform better. Environmentally, however, higher index values are associated with decreased animal performance—pigs suffering environmental (pathogen) challenges will mount an immune response resulting in higher index values but poorer performance. Therefore, taking an index value, as a single entity, may not be appropriate as the index phenotype confounds conflicting genetic and environmental effects. There are two solutions to this problem.
Firstly, in the case of limited data the index may be rejected and individual trait measures used, ie acute phase protein levels or white blood cell count (as this was unrelated to the performance at the individual pig level). As an extension to this solution, the proportion of mononuclear cells positive for NK, B cell and monocyte markers may be used to statistically pre-correct performance for the effect of any environmental challenges
Secondly, if sufficient data exists on related animals, the index value for each animal may be decomposed using a statistical technique known as Best Linear Unbiased Predictor (BLUP) into a genetic and environmental component. BLUP is a standard technique used by animal breeders to disentangle genetic and environmental effects on performance, in order to identify animals with the best genotypes. The environmental component can then be used to pre-correct performance traits for environmental challenge effects, and the genetic component used along with the corrected performance traits in a selection index describing overall performance.

This second strategy should efficiently use both attributes of the index and produce pigs better able to various environmental challenges.

### EXAMPLE 2

Demonstration of the Validity of Using WBC counts to Improve the Performance and Efficiency of Progeny in Commercial Environments.

Below data is provided demonstrating that the technique of using WBC counts or WBC QTL within a specific-pathogen-free environment to genetically improve performance and efficiency of progeny performing in a commercial environment works in practical situations.

Immunological Traits as Predictors of Performance Traits for Progeny

A total of 92 male pigs undertook a standard performance test on a specific pathogen-free farm. At the end of test (91 kg), white blood cell (WBC) counts were performed on all pigs. Standardised WBC count (SWBC) for each pig was estimated as the deviation of the individual WBC from the mean of its contemporaneous pigs. Five pigs were chosen at random to be used as sires. Progeny of these sires were born and reared on two farms (farm 1=252 progeny, farm 2=138 progeny), and the performance of these progeny was evaluated on a standard performance test. The progeny traits of daily gain and fat depth at 91 kg were obtained. The utility of SWBC as a predictor of progeny performance was evaluated by (i) regressing progeny traits on sire SWBC and (ii) calculating the correlation coefficient sire SWBC and the progeny family mean. In these analyses the individual pig sex and weight at the start of test, and the weekly batch number were also fitted in the statistical analysis. The trait of lean gain was not calculated, however improved lean gain is indicated by a combination of increased daily gain and/or decreased fat depth.

Results

The results are shown in Table 10. The regression of progeny fat depth on sire SWBC was highly significant on both farms, with increased sire SWBC associated with decreased progeny fat depth. This is also indicated by the strong negative correlations between progeny mean fat depth and sire SWBC. The correlation between progeny mean daily gain and sire SWBC was positive, i.e. in the predicted direction. These results indicate that sire SWBC is predictive of performance: increased sire SWBC is associated with significantly decreased progeny fatness and a trend towards increased daily gain, which together indicate enhanced lean gain, high efficiency and high performance mammals.

TABLE 10
Relationship between progeny performance and sire
WBC (NS not significant, ** p < 0.01, *** p < 0.001)

| | | Daily gain | Fat depth at 91 kg |
|---|---|---|---|
| Farm 1: | Regression | NS | *** |
| | Correlation | 0.38 | −0.68 |
| Farm 2: | Regression | NS | ** |
| | Correlation | 0.20 | −0.70 |

Discussion

This experiment tested the prediction that WBC counts can be used as predictors of progeny performance under commercial conditions. The data presented here is evidence of the validity of this prediction: increased sire WBC counts are associated with desirable changes in progeny performance for both daily gain and fat depth. Therefore, sire WBC counts may be used as a selection criterion to improve progeny performance. Increased sire WBC will also be associated with enhanced efficiency in these pigs.

1. A method of assaying for mammals having a high innate immunity level, said assay comprising the steps of:
(i) assessing the total white blood cell count of the mammal or at least one of the mammal's parents and/or the acute phase protein level of the mammal or at least one of its parents or genetic markers indicative of the white blood cell count of the mammal and/or the acute phase protein level of the mammal;
(ii) comparing the measurements so obtained with equivalent measurements from other mammals of the same breed wherein measurements higher than mean equivalent measurements from mammals of the same breed indicate a high innate immunity level.

2. A method as claimed in claim 1 including the step of assessing the proportion of mononuclear cells positive for NK, B cell and monocyte markers wherein a proportion higher than the mean proportion for mammals of the same breed indicates an increased risk of reduced performance of the individual due to infection.

3. A method as claimed in claim 1 wherein the mammal is a pig.

4. A method as claimed in claim 1 wherein all sample compared are extracted from the mammals within 24 hours of each other.

5. A method as claimed in claim 1 wherein the assessment of the innate immunity levels of the mammal is performed less than twenty four hours from extraction of the sample being assessed from the mammal.

6. A method as claimed in claim 1 wherein the acute phase protein is alpha-1 acid glycoprotein or serum amyloid A.

7. A method of assaying for a breed of a type of mammal having a high innate immunity level comprising the steps of performing the method as claimed in claim 1, calculating an average innate immunity level of mammals within a single breed and comparing the average innate immunity level obtained to equivalent values obtained for other breeds of the mammal.

8. An assay to create a generalized immunity index for a mammal by testing the total white blood cell count of the mammal or at least one of the mammal's parents, assessing the proportion of mononuclear cells positive for NK, B cell and monocyte markers and combining these values.

9. An assay as claimed in claim 8 wherein the generalized immunity index is calculated using the following formula:
Index=WBC/(s.d. WBC)+(NK prop)/(s.d. NK prop)+(B prop)/(s.d. B cell prop)+(Monocyte prop)/(s.d. monocyte prop)
wherein—“WBC” is the total white blood cell count, “s.d.” is the standard deviation of a variable, and “prop” is the proportion of mononuclear cells positive for a certain marker.

10. An assay as claimed in claim 8 wherein high generalized immunity index values are associated with mammals having a high innate immunity, compared to mean innate immunity levels for mammals of the same breed.

11. A kit for assessing the innate immunity levels of a mammal comprising means for testing the total white blood cell count of the mammal or at least one of the mammal's parents and/or means for testing the acute phase protein level of the mammal and/or at least one of its parents, and/or means for testing genetic markers indicative of the total white blood cell count of the mammal or means for testing genetic markers indicative of the acute phase protein level of the mammal and means for comparing these values with a standard being the mean value for equivalent measurements for value for equivalent measurements for mammals of the same breed as the mammal being assayed.

12. A kit as claimed in claim 11 comprising means for comparing the values of the total white blood cell count of the mammal or at least one of the mammal's parents and/or the acute phase protein level of the mammal or at least one of the mammal's parents or genetic markers indicative of the white blood cell count of the mammal and/or genetic markers indicative of the acute phase protein level of the mammal with a standard thereby determining the innate immunity level of said mammal wherein the standard is the mean value for equivalent measurements for mammals of the same breed as the mammal being assayed.

13. A kit for assessing the generalized immunity index of an animal, comprising means for testing the total white blood cell count of the mammal and the proportion of mononuclear cells positive for NK, B cell and monocyte markers, means for combining these values and means for comparing these values with a standard thereby determining the generalized immunity index value for said mammal wherein the standard is the mean value for equivalent measurements for mammals of the same breed as the mammal being assayed.
